(19) United States
(12) Patent Application Publication
(10) Pub. No.: US 2006/0154350 A1
(43) Pub. Date: Jul. 13, 2006

(54) Method of production of biodegradable lactic acid polymers and the use of lactic acid polymers produced using such a method

(76) Inventors: Vambola Kolbakov, Taevaskoja (EE); Allan Nurk, Tartu (EE); Jaan Simisker, Tartu (EE); Aida Simisker, Tartu (EE)

Correspondence Address: BIRCH STEWART KOLASCH & BIRCH, PO BOX 747, FALLS CHURCH, VA, 22040-0747, US

(21) Appl. No.: 10/472,445

(22) PCT Filed: Sep. 13, 2001
(86) PCT No.: PCT/EE01/00007

§ 371 Date: Nov. 17, 2005

(30) Foreign Application Priority Data
Mar. 23, 2001 (EE) P200100181

(51) Int. Cl. C12P 7/56

(52) U.S. Cl. 435/139

(57) ABSTRACT

The invention relates to the use of lactic acid and its esters produced upon microbiological fermentation of organic substances, particularly cereal starch, for producing biodegradable lactic acid polymers and use of the biodegradable polymer produced using such a technique. The areas of application of the invention include food processing and chemical technology. The fields of usage of the invention include the production of starch, phytoprotein, lactic acid and its derivatives, including its salts, esters and biodegradable polymer (PLA).

### FIELD OF THE INVENTION

The invention relates to the usage of organic substances, particularly lactic acid obtained by microbiological fermentation of cereal starch and esters of said acid, for production of biodegradable lactic acid polymers, and the usage of the biodegradable polymers produced in this way. The invention also relates to food processing and chemical technology. The application areas of the invention are the production of starch, phytoproteins, lactic acid and its derivatives, including its salts, esters and biodegradable polymers (PLA).

### STATE OF ART

Plastics with long decomposition period are a major source of environmental pollution. The main part of such pollution comes from products that are designed for one-time consumption and have a short consumption cycle, which so far have mostly been made from oil plastics that are not easily decomposed in nature. The reduction of pollution by recycling use is mostly economically non-expedient and often impossible in terms of hygiene. A real solution would be using biodegradable polymers, which adjustable decomposition period. One of the most effective materials of this type is the lactic acid polymer, i.e. polylactate, which until now has been used primarily in medical products, but not for consumer goods.

A well-known method for producing the lactic acid for its polymers is microbiological synthesis that uses lactic acid fermentation of pre-processed starch with lactic acid bacteria such as Lactobacillus amylophilus and L. amylophorus or Lactobacillus bulgaricum and L. delbrueckii. The raw materials starch-rich, crops have been used, whereas corn and manioc (cassaya) being the best respectively among cereals and tuber cultures. Separation of starch from and its purification from the crop and tubers is technologically known. Since most of the lactic acid bacteria cannot utilize starch as a growth substrate, it must be converted into sugar (glucose) in advance using appropriate enzymes, which is performed in the following order: starch→dextrin→maltose→glucose, and after that the fermentation process is initiated. The lactic acid produced through fermentation is separated from the solution either in the form of calcium salts by sedimentation, by extraction with anon-polar solvents, or electrodialysis, and if necessary, purified using esterification and selective membranes (WO 9 950 345, U.S. Pat. No. 4,885,247, EP 0 657 542 A1, U.S. Pat. No. 5,053,485, U.S. Pat. No. 5,503,750, U.S. Pat. No. 5,723,639, CN 1 050 203, JP 4 359 014 and WO 9 509 879). Residues of the fractionation and fermentation processes of grain can be used as forage additives.

In connection with the development of the corresponding technology, lactic acid polyester that has been used mostly in exclusive medical devices is turning into a base material of consumer plastics. The most common name of lactic acid polyester in relation with its production technique over lactide is polylactic acid (PLA) (Witzke, D. R.; Narayan, R., Kolstad, J. J. Macromolecules, 1997, 30, 7075). Optically pure lactic acid is preferred for PLA synthesis, and the sole industrial manufacturing technique of said lactic acid is the microbiological production from sugars and their blends. Considering the volumes of lactic acid necessary for economically expedient production of PLA, the main raw material available in the necessary quantity is starch, particularly cereal starch.

Considering the aforesaid, a common production line from cereal to PLA plastic is being designed. So far, the technology of production of PLA is divided into three separate plants: one for production of fermentable sugars, another for fermentation of sugars into lactic acid, and the third for PLA synthesis. A common, integral technological production scheme for producing PLA from cereal has not been patented yet, and the separateness of the production lines does not enable co-ordinated use of the resources, primarily in terms of water, energy, and mineral and nitrogen compounds found in cereal.

According to the most common cereal processing schemes, the fibre-rich outer layer of grains is removed at first—fractionation of bran. Upon further processing of cereal, fractions of starch and protein are separated. Such plants have been founded already since 1835 (Godon et al., Qual. Plant Foods Hum. Nutr., 1983 33:161). So far, water has been used upon fractionation (Fellers, In Industrial Uses of Cereals, Ed. Y. Pomeranz, American Association of Cereal Chemists, St. Paul, Minn., 1963 207), although an alternative method that includes ethanol as the separation medium has been patented (U.S. Pat. No. 5,851,301).

The problem in connection with starch plants is large amount of biogene-rich wastewater generated in the course of fractionation (up to 4 m³ per one ton of starch), which must be separated for producing marketable products (starch, gluten, forage), and the solute biogenes have to be extracted in order to reduce environmental pollution. Usually, wastewater is evaporated and dry biogenes are added for example, to the forage fraction. Evaporation of the water consumes a lot of energy.

Industrial fermentation of lactic acid has been practiced for over a century. The first lactic acid plant where sugars were fermented into lactic acid was founded in 1883 in the USA (Avery Lactate Company, Littleton, Mass.). To make feed that is suitable for lactic acid fermentation, sugar and sugar manufacturing residues (molasses), milk whey and partially or fully saccharinized starch have been used. In industry, the most commonly used producer strains come from the genus Lactobacillus, of which the most widely used strain for producing lactic acid is L. delbrueckii NCIB 8130. The production of optically pure L(+) or D(−) lactic acid for synthesising PLA requires a high level of microbiological purity, because in addition to the general threat of microbial contamination, even other producers of lactic acid may impair the optical purity of lactic acid. The growth temperature of the most commonly used industrial lactic acid bacteria is 42-45° C., which does not preclude from the threat of microbial contamination. Alternative producers of lactic acid include the strains of the Bacillus family (U.S. Pat. No. 5,079,164; Payot T. et al. Enzyme and Microbial Technology 24:191-199, 1999; EP 0 770 684; Ohara H. Yahata M. Journal of Fermentation and Bioengineering 1996, Vol 81 Iss 3 pp 272-274).

Lactic acid is marketed in the form of an 80-percent aqueous solution or to a lesser extent, in the form of salts. Since in the plants that produce lactic acid, the final concentration of lactic acid in the fermentation medium is usually not higher than 10-12%, the production of lactic acid requires a lot of water and energy. In order to make a marketable product, 80-100% of the water involved in the process has to be separated.

Unification of a starch plant and production of lactic acid, which our invention involves, enables a rational use of wastewater that is generated upon fractionation as well as its biogenes for producing the growth medium upon the production of lactic acid and the use of water generated upon concentration of lactic acid for fractionation of cereal. So far, the schemes of production of starch and lactic acid in physically separated plants have not been able to use such an opportunity (U.S. Pat. No. 5,453,365, U.S. Pat. No. 5,766,439, U.S. Pat. No. 6,087,532, U.S. Pat. No. 6,187,951, WO/95/24498, WO/98/28433 and EP 0 614 983).

Different solutions of fractionation of grains have been described in the following patents: U.S. Pat. No. 4,132,566, U.S. Pat. No. 4,217,414, U.S. Pat. No. 4,494,530, U.S. Pat. No. 4,689,409, U.S. Pat. No. 5,439,526 and U.S. Pat. No. 5,851,301.

Formation of PLA by polycondensation of lactic acid has already been described in 1932 (Carothers, W. et al. J. Am. Chem. Soc., 1932, vol. 54, 761). Since the extension of the PLA chains during the polycondensation is difficult to control, most of the patented schemes set forth synthesis of PLA over lactide, using the so-called “ring-opening” reaction. There is no large-scale industrial manufacturing of PLA yet. High temperature (up to 200° C.) is needed for lactide production and PLA synthesis. Therefore, unification of a PLA production plant with starch and lactic acid plants enables the use of heat generated in the production of PLA for the liquefaction and saccharification of starch.

Patents U.S. Pat. No. 5,723,639, EP 0 614 983, CN 1 102 180 and GB 346 486 describe the purification of lactate over esters of lactic acid.

The most widely used scheme of PLA synthesis requires synthesis of pure and anhydrous lactide as an intermediate. Several production methods have been described, including using vaporised lactic acid, synthesis from pre-polymer, synthesis from PEG-solution or synthesis from salts (U.S. Pat. No. 5,332,839, U.S. Pat. No. 5,453,365, U.S. Pat. No. 6,005,067; Kamm et al., Acta Biotechnol. 20 (2000) 3-4, 289-304).

Upon usage of a lactate, its salts and esters as feedstock of a polymer, the suitable solutions include those that enable a continuous process (U.S. Pat. No. 6,005,067; U.S. Pat. No. 5,453,365).

Several methods are suitable for the polymerisation of PLA, as described in patents U.S. Pat. No. 6,207,792, U.S. Pat. No. 5,543,494, or in articles by Jacobsen et al. Polymer engineering and science, 1999, Vol. 39, N7, 1311-1319, and Schmack G et al. J. of Biotechnol. 86 (2001) 151-160.

Hereinafter we will show that integration of a starch plant, production of lactic acid and its derivatives, and PLA-synthesis into a single production line is both economically and ecologically a new quality, which enables one to produce more rationally and reduce duplicated vaporisation of large amounts of water, preserves raw material and energy sources.

### BACKGROUND OF THE INVENTION

The aim of this invention is to develop a method of production of lactic acid polymers based on the starch received from cereal cultures of the temperate climate zone. These cereal cultures include wheat, rye, triticale, barley and corn, the grains of which contain over 60% starch. The goal is to accelerate the cultivation cycle, i.e. to shorten the current cultivation period of 6-7 days to 2-2.5 days, as well as increasing the optimal temperature of cultivation from the current 44° C. to 52-62° C., which ensures sterility against almost any contaminating microflora. In addition, the method must ensure upon cultivation a lactic acid yield of not less than 85-90% of the theoretical value, high affinity of the producer to fermented sugar in order to ensure a low concentration of residual sugar, easy separability of microbial cells from the cultivation medium, and high prototrophy to reduce dependency on several growth factors. The given requirements can be fulfilled by using thermotolerant and highly productive bacteria.

The invention concerns an integral solution for the production of lactic acid, lactic acid esters, lactic acid salts and plastics based on lactic acid from cereal starch. In single stages of the integral production scheme, the solutions used are usually current, those realised in production and/or described in patents. The novelty of the invention is achieved by the unification of existing technological entities into one original method, while the integrated production cycle enables to save natural resources both in terms of water and energy and reduces share of biogenes in wastewater compared to production of starch, lactic acid and PLA in independent plants—thus, it simplifies abiding by the environmental regulations.

### DESCRIPTION OF THE TECHNOLOGICAL PROCESS

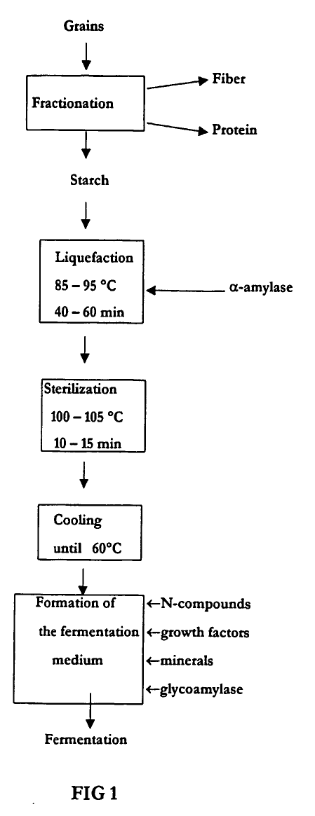

Upon wet milling and fractionation of grains, a starch fraction is produced, from which the most of the fiber and a part of proteins are removed. Using hydrocyclones enables one to separate from the produced starch its purer fraction, the so-called A-starch, which can be marketed as a separate product. In order to produce lactic acid polymers, it is possible to use the purer A-starch as well as so-called B-starch, which contains more additives. In the latter case, the minerals and nitrogen compounds can be marketed as growth factors. Thereafter, starch is liquefied at 75-90° C., then sterilized or pasteurised and used as a fermentation medium to which growth factors are added. In this stage, polysaccharides (i.e. starch) are decomposed into monosaccharides (glucose) using appropriate enzymes and then the produced sugars are fermented into lactic acid (CH₃—CH(OH)—COOH) using corresponding bacteria. This process is schematically described in FIG. 1. Upon the liquefaction and saccharification of starch, the water generated in fractionation of grains is used.

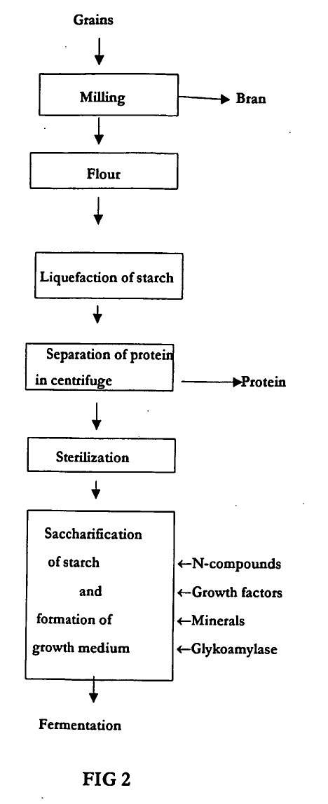

In FIG. 2, an alternative opportunity is described in the event that wet milling of grains and the consequent separation of starch from protein is inexpedient. Pursuant to the latter scheme, it is possible to use 70-75% of the starch of the grains for preparation of the fermentation medium. Fibers are separated from the hydrolysate using centrifugal separation. The supernatant is sterilized and used for producing the fermentation medium, adding yeast extract as a growth factor.

Usually, lactic acid producers require a complex set of growth factors, the exact composition of which has never been described in a uniform way in connection with the strains used. The requirements of the producer used in this patent application in connection with the growth factors are quite completely met by the factors present in the wort and yeast extracts (reference to the patent of Tartu University). The yeast extract is partially replaceable (up to 75%) with hydrolysate of cereal protein without causing substantial deviations in the production and final concentration of lactate. Substitution of the expensive yeast extract with a cheaper phytoprotein is an opportunity to reduce the production cost of lactic acid. Unification of fractionation and fermentation of grains into one technological scheme also enables the use of cereal protein rationally, including the protein, which remains in the fractionation water of starch.

The lactic acid formation speed upon fermentation depends both on the pH of the medium, and actual concentration of lactic acid. The producers used in this patent application enable the fermentation to produce lactate with the concentration of up to 10% upon stabilisation of pH between 5.5-6.0 using CaCO₃ as a neutraliser, but upon application of a relatively long fermentation cycle (up to 120 h). If pH is stabilised between 6.2-6.3 (for example, using NH₃), the fermentation cycle can be reduced to 50-60 hours, while the final concentration of lactate remains between 7.5-12%.

To increase the efficiency of fermentation, the crucial factor is binding the productivity (lactate formation rate per hour per litre) to the final concentration of lactate, which is technologically adjustable by the concentration and composition of growth factors, and pH.

The fermentation process of this patent application is characterised by following parameters:

| | |
|---|---|
| 1. Temperature of fermentation | 52-62° C. |
| 2. Yield of lactate of the glucose used | up to 98% |
| 3. Final concentration of lactate | 8-12% |
| 4. Duration of the cultivation in the batch culture | 50 to 120 hours |
| depending on the pH and the neutraliser | |
| 5. Productivity at lactate concentration of | up to 4 g · l⁻¹ · h⁻¹ |
| 30-400 mM | |
| 6. Productivity of the fermentation cycle | 0.7-1.5 g · l⁻¹ · h⁻¹ |

To ensure efficiency of fermentation, a low content of residual sugars in the medium is crucial. The producers used in this application enable residue-free utilisation of glucose at final concentration of lactate at 8-12%. High temperature of fermentation and the possibility of pH adjustment between 5.5-6.3 enable saccharification to be united with fermentation, preserving suitable conditions for both processes and minimising microbiological contamination.

The separation of lactic acid from the fermentation medium can be carried out using any methods that have been used so far, however preferably using a process which enables continuous technology, for example over esters. The derived lactic acid is directed to the polymerisation stage of lactic acid. Since lactic acid (2-hydroxypropionic acid) contains both the hydroxyl group as well as carboxyl group, it is a typical monomer used in polymerisation and it can form polymeric chains—polyesters. As a result of the process, polylactic acid is formed.

In principle, polymerisation of lactic acid can be achieved in two ways:

1) Direct polymerisation of lactic acid;

2) Polymerisation of lactic acid from its cyclic dimer—lactide.

The molecular mass of polylactic acid produced upon direct polymerisation of lactic acid is low (3,000-4,000) since under the conditions the reaction polymerisation is in balance with depolymerisation. However, the cyclical dimer of lactic acid, i.e. lactide (3,6-dimethyl-1,4-dioxan-2,5-dion) enables the production of (co)polymers that have a high molecular mass.

Synthesis of Lactide

To carry out the synthesis of lactide, a presence of the catalyst is necessary. Metals of the groups 4, 5, 8, 9, 10, 14 and 15 of the periodic system or their compounds, tin and tin compounds, zinc oxide, and salts of alkali metals (except sodium) have been used. The series of activity of metal catalysts is the following: Sn>Zn>Zr>Ti>Al. The reaction is carried out at a higher temperature (150-240° C.) in a vacuum by distilling off the forming lactide (boiling point of mesolactide is 142° C./8 mmHg, melting point 116-119° C.).
Polymerisation of Lactide

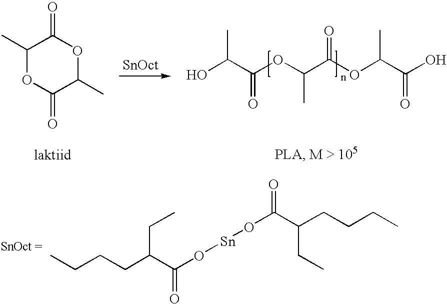

The polymerisation of lactide is performed at a high temperature (˜150-250° C.) in the presence of catalysts like SnCl₄, tetraphenyl tin and Sn-2-ethylhexanate (SnOct), different metal alkoxides like magnesium ethoxide, aluminium isopropoxide, zinc or tin butoxide, zirconium propoxide, tin dibutyl- or tributyl metoxide. The most commonly used catalyst is nevertheless Sn-2-ethylhexanate (SnOct), a fast polymerisation catalyst, which does not cause substantial racemisation at high temperatures.

Polymerisation driven by this catalyst occurs through a non-ionic insertion mechanism.

Optimal concentration of the catalyst depends on the polymerisation temperature and time as well as melting point of the derived polymer. In the presence of water, high molecular weight polylactic acid is thermodynamically unstable and depolymerisation takes place.

By varying the conditions of polymerisation, it is possible to synthesise polylactic acid with different properties (e.g. change the glass transition temperature of the polymer, synthesise amorphous, semicrystalline or crystalline, straight- or branched-chain polymers).

It is also possible to add other monomers and polymers in the course of polymerisation as well as derive copolymers. Polymerisation reaction is of first order both in relation to the monomer and catalyst.

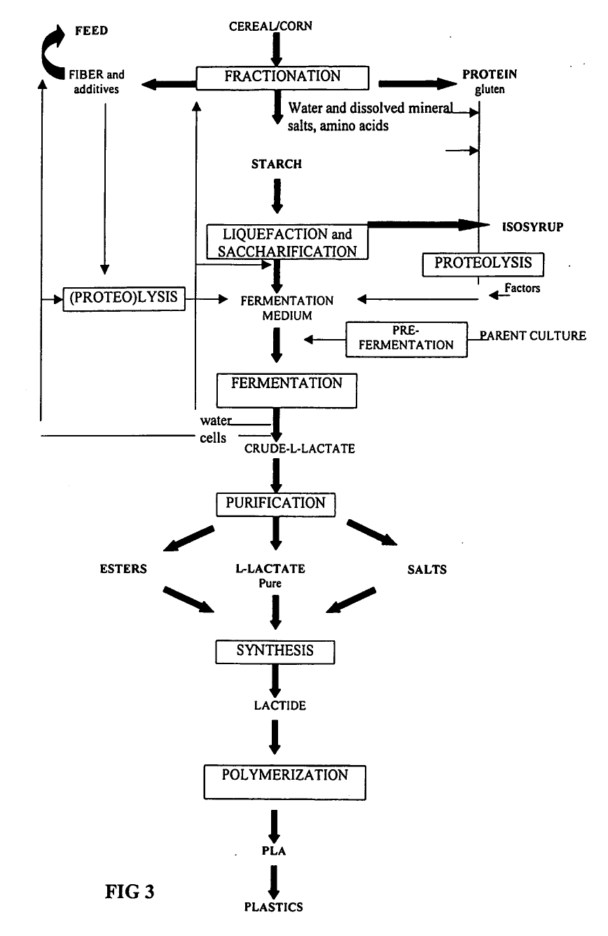

Unification of a starch plant and production of lactic acid as indicated in FIG. 3 enables rational utilisation of residual water generated upon fractionation of starch and its biogenes for producing growth medium for production of lactic acid, and the water released upon concentration of lactic acid for fractionation of cereal meal. Current production schemes of starch and lactic acid in physically separate plants have cannot use this opportunity. Unification of production of lactic acid with a starch plant enables one to abandon the final drying of starch and protein fractions, which makes starch and protein cheaper than upon purchase from starch production plants.

Unification of fractionation of cereal with fermentation and purification of lactate and polymerisation enables the usage of generated water for fractionation of grains. For fractionation purposes, in principle, any earlier patented solution (referred to above), which enables the derivation of pure A-strach and protein, by separating fraction of fibers from them, is suitable.

In our scheme, liquefaction and saccharification of starch is preferably performed enzymatically, but acid hydrolysis cannot be excluded. Lysis of proteins and peptides is preferably performed with proteases.

Saccharification and proteolysis can occur either in special reactors or be partially or fully performed in fermentation tanks.

Fermentation is preferably performed in a semi-continuous process where the establishment and pre-fermentation of mother culture is a continuous process, but main fermentation is performed in batch fermenters. The derived lactic acid is neutralised using a suitable base (Ca²⁺, NH₄⁺, Na⁺, K⁺) or organic bases.

Usable producers include microorganisms that produce pure L(+)- or D(−)-stereoisomers of lactic acid, but the preferred strains are those producing L(+)-lactic acid since L-lactate and the products derived thereof have wider field of applications. Of the latter, the strains of the genus Bacillus with high temperature tolerance and low need for growth factors are preferable.

Usage of temperature-tolerant strains which growth optimum exceeds 55° C. enables one to abandon the usual sterilization of fermentation equipment by high-pressure steam. Of such producers of lactic acid, the most preferred are microaerophile bacilli that provide more cell mass in the pre-fermentation in microaerophile conditions and thus, increase the productivity of main fermentation. Substitution of lactobacilli with oxygen-tolerant bacilli enables one to control the number of cells in the mother culture better, using microaerophile pre-growth.

Upon separation of lactate from the fermentation medium all existing technological solutions, which are adjustable into an integral production scheme with repetitive use of water, such as evaporation, pervaporation, electrodialysis, and reverse osmosis, are usable. The maximum effect achieved by technologies that involve concentration of lactic acid by evaporation.

To derive lactate that is sufficiently pure for polymer synthesis, our scheme prefers purification over lactic acid esters.

The most widely used scheme of synthesis of PLA requires synthesis of pure anhydrous lactide as an intermediate. Several methods have been utilized for deriving it, like formation from evaporised lactic acid, synthesis from prepolymer, synthesis from PEG-solution or synthesis from salts.

Upon the usage of lactate, its salts and esters as the source materials of polymer, the suitable solutions are those including a continuous process.

Several of the methods described above are suitable for polymerisation of PLA.

The scheme of integration of production presented in this application contains several advantages compared to a separately operating starch plant, lactic acid and its derivatives plant and PLA polymer plant.

### EXAMPLES OF IMPLEMENTATION

### Example 1

Wheat flour was suspended respectively in distilled water and in the water released upon concentration of the fermentation medium (the distillate of the medium used for homofermentative fermentation of lactic acid with Bacillus coagulans). The suspensions were allowed to sediment, which resulted in clearly recognisable gluten zone (upper) and fractions of starch and fibers. In two series of experiments, microscopic analysis of the fractions did not indicate any differences.

### Example 2

Fermentative Hydrolysis of Starch in the Environment of the Water Utilized for Fractionation of Cereal.

The water utilized for fractionation was considered to be the water which was derived from suspending cereal (wheat) flour in water for 20 minutes and from which fiber, starch grains and protein particles were separated by centrifugation at 2,000 g for 15 minutes. Utilising said water, suspension of starch was prepared, the full hydrolysis of which would yield an 800 mM aqueous solution of glucose. Distilled water was used as a control solution. Starch (200 g) was liquefied with industrial enzyme alpha-amylase (Nervanase BT) and saccharinized with glucoamylase (Rakvere Distillery, Estonia). To liquefy starch with alpha-amylase, the temperature was increased to 80° C. in 40 minutes and incubated at this temperature for 10 minutes. Independent of the utilized water, a liquefied dextrose aqueous solution with DE=17-20 was derived. The liquefied starch solution was then cooled down to 58° C. and glucoamylase was added. It was incubated at this temperature depending on the amount of glucoamylase added for 3-10 hours up to DE=97-98. The duration of saccharification did not depend on the water utilized. The aforesaid hydrolysate was used to make the feed for the producer of lactic acid.

### Example 3

Fermentative Hydrolysis of Gluten in the Water Used for Fractionation of Cereal

The water utilized for fractionation was considered to be the water which was derived from suspending cereal (wheat) flour in water for 20 minutes and from which fiber, starch grains and protein particles were separated by centrifugation at 2,000 g for 15 minutes. Then, a 12-percent aqueous solution of gluten was prepared using fractionation water or distilled water for that purpose. In both cases, the pH was adjusted to 6 and the suspension incubated at 47° C. For hydrolysis of gluten, an industrial protease complex ‘Flavourzyme’ (NovoNordisk, Denmark) was used at 50 units per gram of protein. Independent of the utilized water, 33% of the possible theoretical yield (determined by ninhydrin) was released as free amino acids after 30 minutes of exposition. Qualitative analysis of amino acids using thin layer chromatography did not indicate any differences in the amino acid composition of the lysate. The aforesaid hydrolysate was used to make the feed for the producer of lactic acid.

### Example 4

Fermentation of Lactate Upon Wholemeal Feed

The starch of the wholemeal flour suspension was hydrolysed to glucose (15.2%), using enzymological processing with alpha-amylase and glycoamylase. Using Novo-Nordisk Flavourzyme (5 u/g, 50° C., 20″) for that purpose, the grain proteins were converted into a blend of amino acids to the extent of 30% in the initial stage of liquefaction of starch. According to the ninhydrine reaction, the total content of amino acids was 1.2%. The derived feed was clarified using centrifugation and the supernatant was fermented with Bacillus coagulans. 96% of the fermented glucose was converted into L-lactate, which accumulates in the fermentation medium as ammonium lactate, resulting in the final concentration of lactic acid of 9.9%. Additional sources of mineral substances and nitrogen compounds were not used.

Cultivation was carried out in anaerobic conditions by mixing at 60 rpm, 57° C., pH value in the stationary regime at 6.3, NH₄⁺ was used as a neutraliser. The duration of the cultivation cycle was 42 hours. After the end of fermentation, the cells were centrifuged out and the ammonium lactate in the supernatant was concentrated by distillation to 80%. The distilled water was condensed and collected for following experiments.

### Example 5

Gluten as the Source of N-Compounds and Growth Factors

a) In the proof test, the feed used as the fermentation medium, was composed by adding 800 mM glucose and gluten (15 g per litre) lysed according to Example 3, and mineral salts to distilled water. The producer of lactic acid used was thermophilic isolate of Bacillus coagulans.

The composition of mineral salts was the following:

| | | |
|---|---|---|
| KH₂PO₄ | 0.2 | gl⁻¹, |
| MgSO₄.7H₂O | 0.2 | gl⁻¹ |
| MnSO₄.4.5H₂O | 10 | mgl⁻¹. |

CaCO₃ was used as a neutraliser. 40% of the glucose was used upon fermentation and into lactate with 94% yield, whereas the concentration of the latter reached 5.4% (in relation to lactic acid).

b) In the following fermentation, upon composing of the feed using the same additives to the fractionation water of grains (as described in Example 2) was used. The feed was used as the fermentation medium for producing lactic acid by the Bacillus coagulans strain. CaCO₃ was used as a neutraliser. Upon fermentation, 58% of the glucose was utilized, which was transformed in 94% into lactate, and the concentration of which in the medium rose to 7.6% (in relation to lactic acid).

c) Adding 5 g of yeast extract per litre of the final fermentation medium resulted in full utilisation of glucose in the fermentation medium, the final lactate concentration of 10.1% and a yield of 90.2% of the glucose utilized.

d) Full utilisation of glucose and final lactate concentration of 10.1% was derived from the substitution of neutralising calcium carbonate with an aqueous solution of 4 M ammonia.

### Example 6

Upon substitution of the yeast extract described in Examples 5c and 5d with a lysate of cells of Bacillus coagulans (derived by proteases Flavourzyme and Neutrase, NovoNordisk, Denmark), 97.2% of the glucose directed to the feed was spent, fermenting 90.2% of sugars into lactate with final concentration of 9.6% (related to the lactic acid).

### Example 7

Polymerisation of Lactate

The derived 800 g of 80% aqueous solution of L-lactate was heated under continuous stirring and argon flow until the water was separated, after which the catalyst was added. The temperature of the reaction medium was increased up to 180° C. and the pressure in the system was lowered to about 6 millibars. Under optimal temperature and pressure conditions a vitreous mass with very good transparency was produced, which was then cooled down to room temperature in an argon flow. The amount of formed mass was 386 g. When melted, the produced polymer could be drawn into long threads (over 10 metres), or used for coating paper sheets, making them waterproof.

Use of Lactic Acid Polymer

The derived pure lactic acid polymer is seldom used in consumer goods due to its brittleness. Its properties can be modified according to the planned application, the characteristics of the environment in the place where the object happens to be used, and where it should be decomposed. Thus, it is necessary to choose the polymer whose properties fulfil its functions as a consumer product and then decompose within the required time in the planned environment.

In order to improve some physical properties of lactic acid polymers, including flexibility, toughness and impact resistance, and for modification of its crystallinity and biodegradability as requested, several additives are used for manufacturing final plastics both upon co-polymerisation with lactic acid (for example, lactic acid with aliphatic and/or aromatic dicarboxylic acid by mediation of oligoalkyleneoxide or lactic acid together with glycol and dicarboxylic acid or lactic acid with polyuretan or aromatic polyester, etc.) as well as for forming physical blends with a lactic acid polymer, for example, blends with other polymers (polyethylenglycol, polypropyleneglycol, polybutylene ether or their blends).

As a thermoplastic polymer, the lactic acid polymer can be used as a material for packing any formed products. One can pack foodstuffs, medical products, items and constructions made from the given polymer. Among other things, it is a suitable material for the base plastic of dipers, because lactic acid polymer is not a favourable growth medium for microbes and thus, it is safer than plastics that are based on cellulose. The lactic acid polymer is easily modifiable into microfibres and can thus be used in textiles which by their qualities such as low binding of humidity, fast drying, no wrinkles, low flammability, good coverage, washability and pleasant weft, can be used to replace natural materials such as silk and cotton. It can also be used for making robes.

The pure, additive-free and unmodified lactic acid polymer is usable in medicine for temporary bandages and supports as well as a self-soluble immobilisation base of locally administrable analgesics, antibiotics and other medical products.

Lactic Acid Polymer and Environmental Protection

The pure, additive-free and unmodified lactic acid polymer is a decomposable material which is decomposed at a temperature of over 57° C. within several days to a couple of weeks, depending on the decomposition technology. The lactic acid polymer decomposes without composting it, but it takes a lot more time.

Modified lactic acid products that are designed for external use and which are to be used at different levels of duration of consumption and biodegradability can be decomposed within a period ranging from a few months up to a couple of years, this goes for in nature as well. Pursuant to one's request, such a plastic of the lactic acid polymer can be designed to be degradable by humidity and/or soil microbes. The plastic can be burnt, because it burns emitting little smoke and without emitting any poisonous gases. The sole burning products are carbon dioxide and water.

1. A method for producing the biodegradable lactic acid polymer from cereal starch characterized in that the source materials used include cereals such as wheat, rye, triticale, oat, barley or corn, wherein the microbiological synthesis of lactic acid is carried out with thermotolerant bacteria, using components of cereal as sources of mineral and nitrogen compounds and in order to produce biodegradable lactic acid polymers, a single integrated production cycle starting from cereal and finishing with PLA is used.

2. A method according to claim 1 characterized in that upon fractionation of cereal into fractions of starch, protein and fibre, the water released upon concentration, estrification and PLA-synthesis is utilized.

3. A method according to claim 1 or 2 characterized in that the water which is released upon fractionation of cereal into fractions of starch, protein and fiber is used for formation of the fermentation medium.

4. A method according to claim 1 characterized in that the water used for fractionation of cereal and partially the protein fraction is utilized for production of lactic acid as the source of N-compounds, vitamins and mineral salts.

5. A method according to claim 1 characterized in that the starch, the sugars dissolved in the water utilized for fractionation of cereal and the starch remaining into the protein fraction, are used as sources of the sugar fermented into lactic acid.

6. A method according to claim 1 characterized in that the starch contained in the water used for fractionation as well as the starch in the protein fraction, depending on the microorganism used before fermentation, are liquefied or liquefied and saccahrinized.

7. A method according to claim 1 characterized in that the saccharification and liquefaction of starch and the starch dissolved and remaining in fractionation waters occurs in the same reactor.

8. A method according to claim 1 characterized in that the liquefaction and saccharification of the starch remaining in the protein fraction occurs parallel to proteolysis of proteins, for which a separate reactor is used, or it is carried out in the reactors of liquefaction and saccharification of starch and it may continue upon fermentation.

9. A method according to claim 1 characterized in that the protein remaining in the fractionation water of grains, the starch fraction and the protein fraction, is subjected to proteolysis upon production of the fermentation medium.

10. A method according to claim 9 characterized in that the proteolysis of proteins occurs with a mixture of peptidases and carboxypeptidases, the optimal pH of the proteolysis of which remains between 5 and 7 and the temperature-tolerance of which is up to 55 degrees.

11. A method according to claim 1 characterized in that the bacterial mass generated in the course of fermentation is used as an additive in production of concentrated forage from fiber and/or from the protein fraction or after a preliminary proteolysis upon production of the fermentation medium, as a source of N-compounds and growth factors.

12. A method according to claim 1 characterized in that lactobacilli can be replaced by oxygen-tolerant bacilli.

13. A method according to claim 1 characterized in that in the said cycle, all the strains of lactobacilli and bacilli in which ferment glucose are used, but the preferred strains are little auxotrophy and have high temperature-tolerance.

14. A method according to claim 1 characterized in that the separation of lactate and purification from the medium is carried out according to any developed scheme that is adjustable to repetitive utilization of the water integral production scheme according to claim 2.

15. A method according to claim 1 characterized in that for deriving cyclical esters of lactate necessary for synthesis of PLA, is springed from lactate esters or prepolymer or lactate salts or a combination of the abovementioned methods.

16. A method according to claim 1 characterized in that the thermal energy of water released upon estrification and polymerisation polymerization and cooling waters is utilized for liquefaction and saccharification of starch.

17. A method according to claim 1 characterized in that the production of by-products and intermediate products and their ratio is regulated at the optimum level upon reception of marketable products.

18. A method according to claim 17 characterized in that the said products are the following: phytoprotein, starch, fiber, lactic acid and its esters and salts, PLA and the products made from them.

19. Use of lactic acid polymers, which has been produced according to method described in claim 1, for the production of packages, medicinal and food accessories, especially for the production of systems, syringe, implants and dishes that are designed for one-time use and for use in the agriculture, automobile, electronics, fiber and textile industries.
